(19) United States
(12) Patent Application Publication
(10) Pub. No.: US 2025/0120971 A1
(43) Pub. Date: Apr. 17, 2025

(54) NOVEL DRUG TREATMENT OF COGNITIVE IMPAIRMENTS ASSOCIATED WITH SCHIZOPHRENIA

(71) Applicant: FLORIDA ATLANTIC UNIVERSITY BOARD OF TRUSTEES, Boca Raton, FL (US)

(72) Inventors: Claire A. RICE, Jupiter, FL (US); Robert W. STACKMAN, Boca Raton, FL (US)

(21) Appl. No.: 18/292,239

(22) PCT Filed: Jul. 22, 2022
(86) PCT No.: PCT/US2022/038042

§ 371 Date: Jan. 25, 2024

Related U.S. Application Data
(60) Provisional application No. 63/226,382, filed on Jul. 28, 2021.

(51) Int. Cl. A61K 31/506; A61K 31/423; A61P 25/28

(52) U.S. Cl. CPC A61K 31/506; A61K 31/423; A61P 25/28

(57) ABSTRACT

Described herein is a novel method of treating a cognitive impairment in a subject, including administering an SK ion channel activator to the subject and alleviating symptoms of the cognitive impairment, wherein the symptoms originate from a condition selected from the group including a) schizophrenia, b) bipolar disorder, c) an attention-deficit disorder, d) a memory deficiency, e) a verbal learning deficiency, and f) an executive function deficiency. In certain embodiments, the SK ion channel activator is CyPPA. In other embodiments, the SK ion channel activator is chlorzoxazone (CZX).

### CROSS-REFERENCE TO RELATED APPLICATIONS

This international application claims the benefit under 35 U.S.C. § 119 (e) of Application Ser. No. 63/226,382 filed on Jul. 28, 2021 entitled NOVEL DRUG TREATMENT OF COGNITIVE IMPAIRMENTS ASSOCIATED WITH SCHIZOPHRENIA, the entire disclosure of which is incorporated by reference herein.

### FIELD OF INVENTION

This invention relates to a novel use of an activator of small conductance calcium-activated potassium (SK) ion channels and a method of administering a novel drug treatment to patients exhibiting cognitive symptoms associated with schizophrenia, attention deficit disorders, and other cognitive impairments.

### BACKGROUND OF THE INVENTION

Schizophrenia affects approximately 24 million people or 1 in 300 people (0.32%) worldwide. This rate is 1 in 222 people (0.45%) among adults. Attention deficit disorders affect an estimated 2.8% of adults worldwide. This rate has increased significantly in recent years.

The pathophysiology that contributes to the predominant symptoms of psychosis has led to two hypotheses: hyperactive dopaminergic signal transduction and hypofunctional glutamatergic neurotransmission. Central to the glutamate hypothesis is dysfunction of cortical NMDA receptor (NMDAR) activity that shifts the balance of excitation and inhibition towards increased neuronal activity. The consequential alterations of dopaminergic and GABAergic pathways, excessive glutamate release, and oxidative stress contribute to the disease sequelae and precipitate some symptoms of schizophrenia. The hypothesis of hypofunctional NMDARs in schizophrenia predicts that drugs that enhance NMDAR activity may modulate symptoms and enhance cognition in schizophrenia patients.

Small conductance Ca²⁺-activated potassium channels (SK channels) are expressed on neurons throughout the central nervous system and are voltage-insensitive yet activate in response to increases in intracellular Ca²⁺ concentration. SK2 channels regulate neuronal excitability by mediating the medium component of the afterhyperpolarization. Synaptic SK2 channels, positioned within the Ca²⁺signaling domain of glutamatergic synapses, limit glutamatergic postsynaptic potentials and constrain the induction of NMDAR-dependent synaptic plasticity. Thus, in the healthy brain, a negative feedback loop enables synaptic SK channels, activated by Ca²⁺ through NMDARs, to restrict the continued activation of NMDARs; blocking SK channels enhances NMDAR activity. In disease states where irregular neuronal activity promotes behavioral disturbances, SK channel openers or activators can restore neuronal activity and improve behavior.

SK channels act as a brake on synaptic plasticity through a feedback modulation of NMDAR-type glutamate synapses. Homomeric or heteromeric SK channels are comprised of SK1, SK2 and SK3 subtypes. SK channels expressed in several forebrain regions such as the hippocampus limit the encoding of long-term memory.

CyPPA and chlorzoxazone (CZX) are activators of small conductance calcium-activated potassium (SK) ion channels. As an FDA-approved drug, CZX is currently prescribed as a centrally-active muscle relaxant, and a treatment for muscle spasticity. Restoration of motor coordination by CZX is well tolerated, and evidence indicates that side effects are rare.

In healthy controls, blocking SK channels enhances hippocampal-dependent memory, while activating SK channels impairs memory. However, there is a need to determine the efficacy of SK channel drugs to affect the expression of psychotic symptoms in schizophrenia and other models of psychosis and attention deficit.

### BRIEF SUMMARY OF THE INVENTION

A first aspect of the invention is the use of an SK channel activator to treat cognitive impairments in a subject. It is an unexpected advantage of the present invention to use a particular dosage model that combines a dosage and the time of administration to provide the most beneficial effects.

In certain exemplary embodiments, the method further includes alleviating symptoms of the cognitive impairment, wherein the symptoms originate from a condition selected from the group including a) schizophrenia, b) bipolar disorder, c) an attention-deficit disorder, d) a memory deficiency, e) mania, f) a verbal learning deficiency, and g) an executive function deficiency.

In certain exemplary embodiments, the subject is a mammal.

In certain exemplary embodiments, the subject is a human.

In certain exemplary embodiments, the administering step is carried out through an administration technique including at least one of a) intravenous administration, b) oral administration, and c) transdermal administration.

In certain exemplary embodiments, oral administration is performed through an excipient selected from the group including a pill, dissolvable film, tablet, capsule, and liquid.

In certain exemplary embodiments, the SK ion channel activator is an SK2 specific activator.

In certain exemplary embodiments, the SK ion channel activator is a member of the 1,3-benzoxazole drug class.

In certain exemplary embodiments, the SK ion channel activator is CyPPA.

In certain exemplary embodiments, the SK ion channel activator is chlorzoxazone.

In certain exemplary embodiments, the SK ion channel activator is administered in an amount of 1 to 5 times per day.

In certain exemplary embodiments, the SK ion channel activator is administered in intervals of every 2 to 5 hours.

In certain exemplary embodiments, CyPPA is administered at a dose of 70.0 mg/kg.

In certain exemplary embodiments, chlorzoxazone is administered at a dose in an amount selected from a range of 0.24 mg/kg to 750 mg/kg.

In certain exemplary embodiments, chlorzoxazone is administered 3 to 4 times per day.

In certain exemplary embodiments, chlorzoxazone is administered 2 to 5 times per day.

### BRIEF SUMMARY OF THE DRAWINGS

Various exemplary embodiments of the invention will be described in detail with references to the following drawings:

FIG. 1 shows a graph of a CyPPA IP object recognition memory test results measuring the discrimination ratio of control (SAL) and ketamine-impaired mice.

FIG. 2 shows a graph of a CyPPA infusion object recognition memory test results measuring the discrimination ratio of control (SAL) and ketamine-impaired mice.

FIG. 3 shows a graph of chlorzoxazone IP object recognition memory test results measuring the discrimination ratio of control (SAL) and ketamine-impaired mice.

FIG. 4A shows a graph of CyPPA IP trace fear conditioning attention test results measuring the discrimination ratio of control (SAL) and ketamine-impaired mice.

FIG. 4B shows a graph of CyPPA IP trace fear memory test results measuring the discrimination ratio of control (SAL) and ketamine-impaired mice.

FIG. 5 shows a graph of CZX trace fear memory test results measuring the discrimination ratio of control (SAL) and ketamine-impaired mice.

### DETAILED DESCRIPTION OF THE INVENTION

The present invention comprises a novel method of using an SK ion channel activator or opener to treat cognitive symptoms associated with schizophrenia in a subject. In certain exemplary embodiments, the subject is a mammal. In further exemplary embodiments, the subject is a human. In certain embodiments, the method further includes alleviating symptoms of the cognitive impairment, wherein the symptoms originate from a condition such as schizophrenia, bipolar disorder, mania, an attention-deficit disorder, a memory deficiency, a verbal learning deficiency, or an executive function deficiency.

The SK ion channel activator or opener is be administered via methods including but not limited to intravenously, transdermal administration, or orally. In certain embodiments, oral administration is performed through an excipient including but not limited to a pill, dissolvable film, tablet, capsule, or liquid.

In certain embodiments, the SK ion channel activator is an SK1, SK2, or SK3 specific activator. In certain embodiments, the SK ion channel activator is a member of the 1,3-benzoxazole drug class. In additional examples of the method of the invention, the SK ion channel activator is selected from the list including but not limited to CyPPA or chlorzoxazone. The SK ion channel activator is administered about 1 to 5 times per day. In certain embodiments, the SK ion channel activator is administered 2 to 5 times per day. In further embodiments, the SK ion channel activator is administered 3 to 4 times per day.

In exemplary embodiments using CyPPA as the SK ion channel activator, a therapeutically effective amount of CyPPA is administered. In certain exemplary embodiments, CyPPA is administered at a dose of 70.0 mg/kg. In exemplary embodiments using chlorzoxazone, a therapeutically effective amount of chlorzoxazone is administered. In certain exemplary embodiments, chlorzoxazone is administered at a dose ranging from 0.24 mg/kg to 750 mg/kg.

In certain embodiments, the cognitive impairment(s) for which rescue effects are shown are attention, working memory, verbal learning and memory, and executive functions.

In certain embodiments, the cognitive impairment(s) for which rescue effects are shown are temporal lobe-specific episodic memory and attention. It has been surprisingly found that administration of both acute CyPPA and CZX result in rescue of long-term memory and attention.

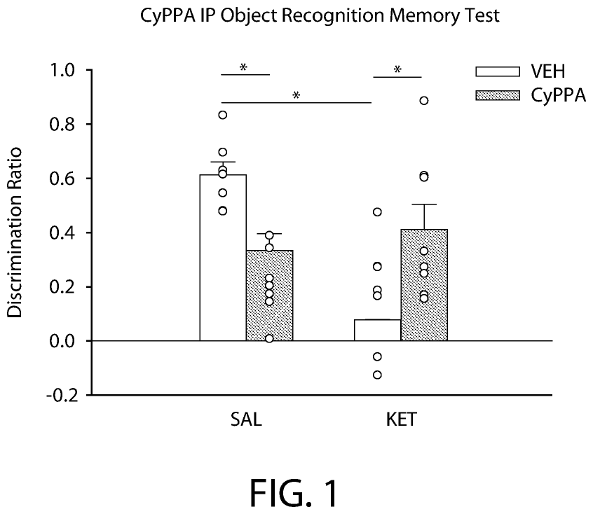

Referring to the Figures, FIG. 1 shows a graph of a CyPPA IP object recognition memory test results measuring the discrimination ratio of control (SAL) and ketamine-impaired mice. Mean±SEM discrimination ratio scores for the object recognition test session presented 24 hours after the sample session. Discrimination ratio is a measure of memory-higher scores indicate better memory. Each mouse received an intraperitoneal (IP) injection of vehicle or CyPPA (15.0 mg/kg) 30 min prior to the sample session. White and gray bars across graphs represent VEH- vs. CyPPA- pre-sample session treated groups, respectively. Memory performance of vehicle (VEH)-treated ketamine (KET) mice was significantly impaired compared to VEH-treated control (SAL) mice, *p<0.001 SAL vs. sub-chronic KET groups. CyPPA treated KET mice performed significantly better than VEH-treated KET mice, indicating SK channel mediated rescue of memory, *p<0.05. A Holm-Sidak multiple comparisons test was performed after group x pre-sample session treatment interaction, SAL/VEH vs. SAL/CyPPA; KET/VEH vs. KET/CyPPA; SAL/VEH vs. KET/VEH. Each filled circle is an individual subject's score.

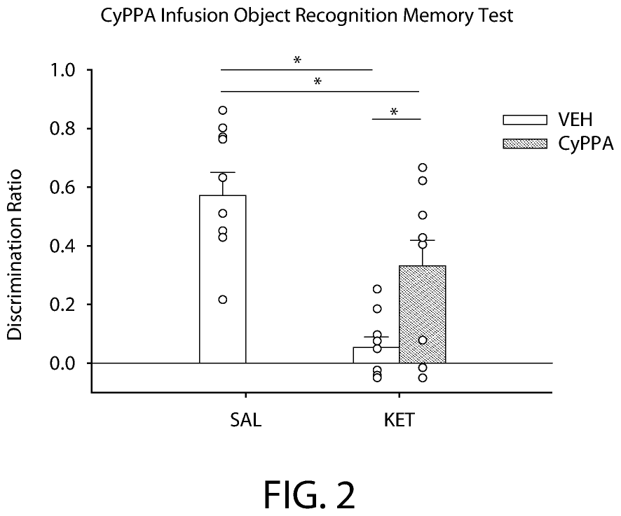

FIG. 2 shows a graph of a CyPPA infusion object recognition memory test results measuring the discrimination ratio of control (SAL) and ketamine-impaired mice. Direct infusion of CyPPA into the hippocampus improved object recognition memory in mouse models of psychosis. Each mouse received a bilateral (0.35 μl/side) microinfusion of VEH or CyPPA (1 μg/μl) immediately prior to a sample session. Recognition memory was tested 24 hours after the sample session. Mean±SEM discrimination ratio for object sessions. White and gray bars across graphs represent VEH-vs. CyPPA-pre-sample session treated groups, respectively. Discrimination ratio scores were significantly different between groups, *p<0.001, SAL vs. sub-chronic KET groups; and a Holm-Sidak multiple comparisons test was performed after group x pre-sample session treatment interaction, *p<0.001, SAL/VEH vs. KET/VEH; and **p<0.05, SAL/VEH vs. KET/CyPPA, and KET/VEH vs. KET/CyPPA. Scores for each subject are represented by the filled circles. Results indicate that direct activation of hippocampal SK2 channels significantly improves recognition memory in the KET mouse model of psychosis.

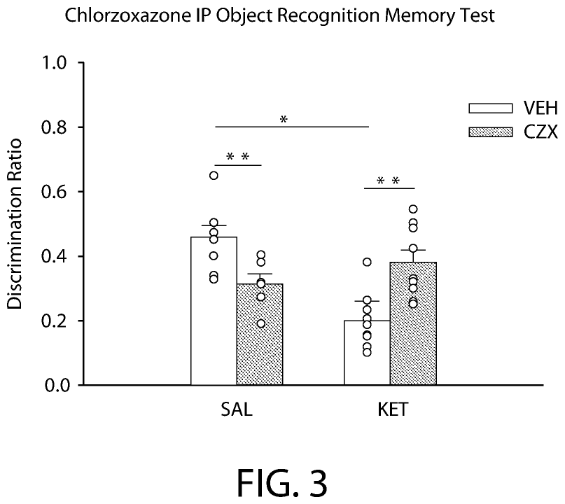

FIG. 3 shows a graph of chlorzoxazone (CZX) IP object recognition memory test results measuring the discrimination ratio of control (SAL) and ketamine-impaired mice. The SK activator, CZX, improved object recognition memory in mouse models of psychosis. As in FIGS. 1 and 2, recognition memory is measured by the discrimination ratio. Mean±SEM discrimination ratio was measured for each group during the test session. White and gray bars represent groups of mice given VEH-vs. chlorzoxazone (CZX, 5.0 mg/kg, IP) before the sample session. Recognition memory was tested 24 hours later. The KET mice given VEH exhibited impaired memory, while the KET mice given CZX exhibited significantly better memory and performed comparably to SAL controls given VEH, **p<0.001, KET/VEH vs. KET/CZX; SAL/VEH vs. KET/VEH. Significant differences in test session discrimination ratio were found between groups, *p<0.05, SAL vs. sub-chronic KET groups; and *p<0.05, Holm-Sidak multiple comparisons test after group x pre-sample session treatment interaction, SAL/VEH vs. SAL/CZX.

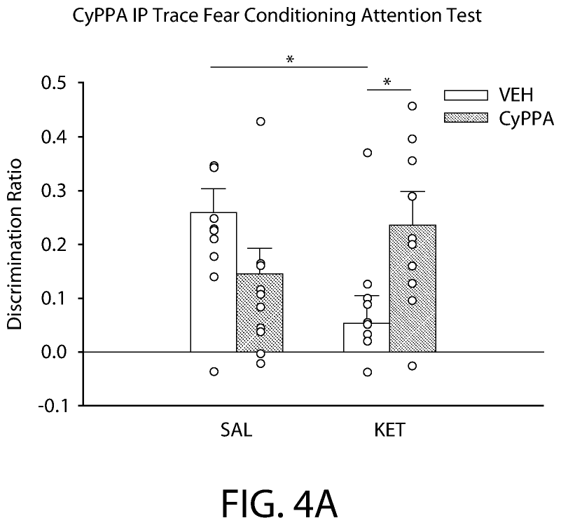

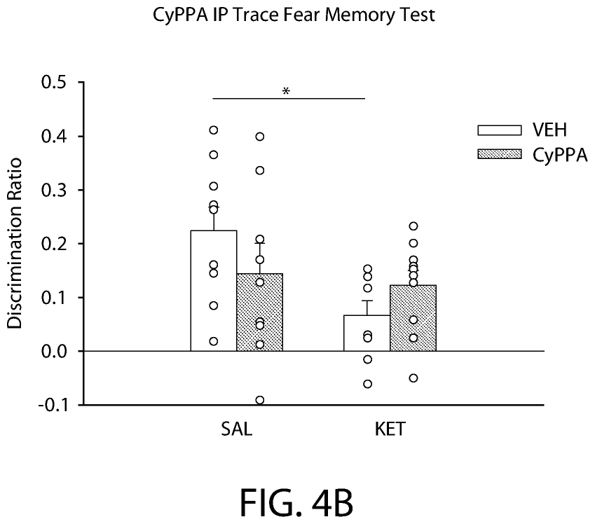

FIG. 4A shows a graph of CyPPA IP trace fear conditioning attention test results measuring the discrimination ratio of control (SAL) and ketamine-impaired mice. The SK2/SK3 activator, CyPPA, improved temporal memory in the ketamine mouse model of psychosis. Each mouse received an IP injection of vehicle or CyPPA (15.0 mg/kg) 30 minutes prior to trace fear conditioning. Temporal memory and attention were inferred from the discrimination ratio; higher value indicates better attention and recall of fear memory. Mean±SEM percent freezing for discrimination ratios were measured for the trace fear conditioning test. White and gray bars across graphs represent VEH- vs. CyPPA- pre-conditioning treated groups, respectively. VEH-treated KET mice exhibited impaired temporal memory compared to VEH-treated SAL controls. However, temporal memory of CyPPA-treated KET mice was significantly better than that of VEH-treated KET mice. The group x pre-sample treatment interaction on discrimination ratio data during the test session was significant, *p<0.05. A Holm-Sidak multiple comparisons test was performed after group x pre-sample session treatment interaction, SAL/VEH vs. KET/VEH, and KET/VEH vs. KET/CyPPA. Discrimination ratios in the test session were equivalent between SAL vs. sub-chronic KET, and pre-sample session treatment groups, VEH vs. CyPPA, as shown in FIG. 4A.

FIG. 4B shows a graph of CyPPA IP trace fear memory test results measuring the discrimination ratio of control (SAL) and ketamine-impaired mice. Significant differences, *p<0.05, between groups were found in discrimination ratios during trace fear test in group and in interaction effects, Holm-Sidak multiple comparisons test after group x pre-conditioning treatment interaction, SAL/VEH vs. SAL/KET. Test session discrimination ratios were equivalent between VEH-treated SAL controls and CyPPA-treated KET mice. These results demonstrate that the SK2/SK3 activator rescued temporal memory and attention in the KET model of psychosis.

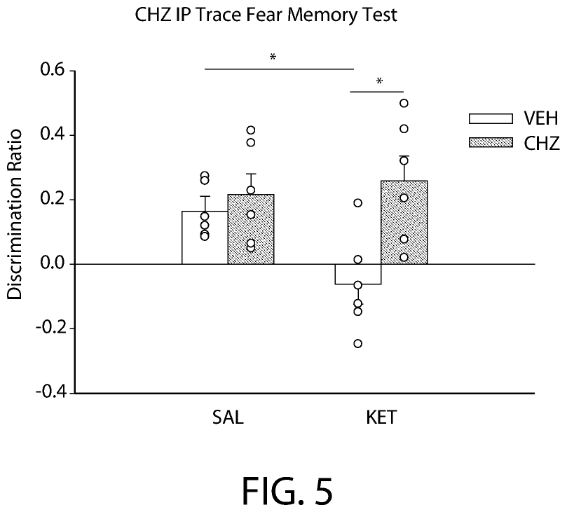

FIG. 5 shows a graph of CZX IP trace fear memory test results measuring the discrimination ratio of control (SAL) and ketamine-impaired mice. The SK2 activator, CZX, improved temporal memory in the ketamine mouse model of psychosis. Each mouse received an IP injection of vehicle or CZX (5.0 mg/kg) 30 minutes prior to trace fear conditioning. Temporal memory and attention were inferred from the discrimination ratio; higher value indicates better attention and recall of fear memory. Mean±SEM percent freezing for discrimination ratios were measured for the trace fear conditioning test. White and gray bars across graphs represent VEH-vs. CZX-pre-conditioning treated groups, respectively. VEH-treated KET mice exhibited impaired temporal memory compared to VEH-treated SAL controls. However, temporal memory of CZX-treated KET mice was significantly better than that of VEH-treated KET mice. The group x pre-sample treatment interaction on discrimination ratio data during the test session was significant, *p<0.05. Holm-Sidak multiple comparisons test after group x pre-conditioning treatment interaction, SAL/VEH vs. SAL/KET. Test session discrimination ratios were equivalent between VEH-treated SAL controls and CZX-treated KET mice. These results demonstrate that CZX rescued temporal memory and attention in the KET model of psychosis.

The invention will be illustrated in more detail with reference to the following Examples, but it should be understood that the present invention is not deemed to be limited thereto.

### EXAMPLES

### Example 1. Systemic CyPPA Improves Object Recognition Memory in a Mouse Model of Psychosis or Schizophrenia

In healthy rodents, blocking SK channels enhances hippocampal-dependent memory (Criado-Marrero et al., 2014; Mpari et al., 2005; Stackman et al., 2002a; Vick, Guidi, & Stackman, 2010), while activating SK channels impairs memory (Hammond, Bond, Strassmaier, Ngo-Anh, Adelman, Maylie, & Stackman, 2006; Mckay et al., 2012; Rice & Stackman, submitted; Stackman Jr. et al., 2008; Vick, Guidi, & Stackman Jr., 2010). Here the efficacy of CyPPA, a SK2 specific activator, was tested on cognitive memory impairments in a schizophrenic mouse model.

### Methods and Materials

KET Model of Psychosis or Schizophrenia-like Symptoms.

Repeated sub-chronic administration of the non-competitive NMDAR antagonist, ketamine (KET) to rodents produces glutamatergic and GABAergic dysfunction and prolonged cognitive deficits like those of schizophrenia (Arslan et al., 2016; Gazal et al., 2015; Ghedim et al., 2012; Nikiforuk & Popik, 2014; Rajagopal et al., 2016; Schumacher et al., 2016). Following a repeated KET treatment (30 mg/kg 2×/day for 7 days followed by a 7-day wash out period, (Rajagopal et al., 2016), male mice exhibited significant and lasting impairments in object recognition memory, and temporal memory and attention impairments assessed in a trace fear conditioning task.

SK Drug Administration. To test the effect of the SK2 specific activator, CyPPA on long-term memory for objects, two KET groups and two SAL groups of male C57BL/6J mice were randomly assigned to receive either VEH or CyPPA (15.0 mg/kg) IP 30 min prior to the sample session of an object recognition memory task. Separate groups received CyPPA (7.5 mg/kg) IP or VEH 30 min prior to the sample session. To improve rigor and reproducibility behavioral testing was conducted by an experimenter blind to the treatment condition and group of the mice. KET mice given CyPPA IP (15.0 mg/kg) spent significantly more time exploring the novel object during the test session, indicating improved recognition memory.

Arena Habituation. Mice were habituated to the arena prior to testing recognition memory. Thirty minutes prior to each arena habituation (AH) session, each mouse was restrained gently and momentarily, given a mock IP injection with a 26-gauge needle, and then placed into an empty polycarbonate cage. Following the delay, the mouse was placed individually into the symmetrical high-walled square arena (37.5×37.5×50 cm, constructed of white ABS) for 10 min. Afterwards, each mouse was returned to the polycarbonate cage and then returned to its home cage. Activity was recorded and automatically scored using Noldus Ethovision XT (version 7.0). Distance traveled was defined as the sum of recorded movement of the center point of the mouse in cm over the duration of the trial. Movement data from the AH sessions was used to match future treatment groups. A one or two-way ANOVA was conducted to confirm that future treatment assignments yielded groups of mice matched for distance traveled and velocity during the AH sessions.

Sample Session. To examine the efficacy of activating SK channels on hippocampal long-term memory in the KET model of schizophrenia or psychosis-like cognitive deficits, an SK channel activator or vehicle (VEH, i.e., placebo) was administered to cohorts of saline (SAL) control mice or ketamine (KET)-exposed mice prior to the sample session of an object recognition task. Ten days after the final SAL or KET injection, mice received an IP injection of a SK channel activator or VEH, and then were placed individually in polycarbonate holding cages. Following a delay, the mouse was placed individually into the familiar arena for 10 min. Mice were permitted to freely explore two identical novel objects that were placed in opposite corners within the arena. The objects, two stainless steel threaded table feet (A and A′), two plastic gorillas (B and B′), or 2 plastic bishop chess pieces (C and C′), each mounted on a clear acrylic base (6×6 cm), had previously been pre-screened for preference and discrimination (Cohen et al., 2013; Hammond et al., 2004), which reduces any potential bias that might be introduced by the objects. The arena and objects were wiped down with 70% ethanol before each session to eliminate any odor cues that were present. Object exploration was defined as time (in s) that the mouse spent with its head oriented toward and within 2-3 cm of the object. The sample session ended after 10 min; at which time the mouse was returned to its home cage. Sample session behavior was video tracked via Ethovision XT and recordings were analyzed off-line to determine object exploration times for each mouse. Mice that did not accumulate at least 30 s. of exploration time per object, or 38 s. exploration time for one object within the 10-min sample session were excluded from the study.

Test Session. Twenty-four hours following the sample session, mice were placed in a polycarbonate cage and then given a 10-min test session in the same arena as the sample session. Each mouse received a needle prick to simulate an IP injection before being placed in the polycarbonate holding cage for 30 min. For the test session, one of the previously encountered objects was replaced with a novel one (e.g., A, B, or C), as described above. To avoid place preference confounding behavioral responses, the objects were randomly replaced for the test session. The arena and objects were wiped down with 70% ethanol before each session to eliminate any odor cues that might affect behavioral responses during the test session.

Statistical Analysis. Object exploration was determined for each mouse based on the manual scoring of behavior from the recorded video files of the sample and test sessions. Scoring was done by experimenters who were blind to the treatment and group of the mice. Object memory was inferred from discrimination ratio scores, calculated for each mouse as the difference in time spent exploring the novel object minus the time spent exploring the familiar object, divided by the total time spent exploring both test objects (TNovel object−TFamiliar object/TNovel object+TFamiliar object). Discrimination ratio scores range from 1 to −1, with ratios greater than zero indicating preferential exploration of the novel object, and ratios less than zero indicating preferential exploration of the familiar object. Higher discrimination ratio values indicate better recognition memory; a score of 0 indicates chance performance. Discrimination ratios were analyzed by a two-factor (group, SAL or KET; treatment, VEH, SK drug) ANOVA. A one-way ANOVA or Student's t-test was conducted to determine whether discrimination ratio scores were significantly different from a chance ratio of 0.

In addition, distance traveled, and velocity was recorded by Noldus Ethovision XT software (version 7.0) and analyzed by two-factor (group, SAL or KET; treatment, VEH, SK drug) ANOVA.

### Results

CyPPA (15.0 mg/kg, IP) Improves Object Recognition Memory in KET Mouse Model. To determine whether CyPPA would mediate rescue of cognitive deficits in KET-mice, cohorts of SAL and KET mice received intraperitoneal injection of CyPPA (7.5 or 15.0 mg/kg) or Cremaphor vehicle (VEH) prior to the sample session. The results demonstrated that KET mice given 15.0 mg/kg of CyPPA pre-sample session exhibited significantly better object recognition memory performance during the test session compared to KET-mice given vehicle (VEH). KET-mice that received 7.5 mg/kg low dose of CyPPA pre-sample showed no rescue of KET-induced impaired object memory 24-h later. Mice that received KET and the low dose CyPPA did not demonstrate exhibit significantly greater exploration of the novel test object than did the KET/VEH-treated mice, indicating that CyPPA produces a dose-dependent rescue of memory in the KET mouse model.

Sample Session. In the 15.0 mg/kg group the results of the two-factor ANOVA (group: SAL vs. KET; pre-sample treatment: VEH vs. CyPPA) indicated that all mice performed equivalently during the sample session. Specifically, analyses of total object exploration time yielded a non-significant main effect of group, F(1, 24)=1.072, n.s., pre-sample treatment, F(1, 24)=2.562, n.s. and group x pre-sample treatment interaction effect, F(1, 24)=0.383, n.s. Measures of distance traveled, and velocity were analyzed to examine whether locomotor performance was affected by the systemic treatment. A two-factor ANOVA on distance traveled during the sample session revealed a non-significant main effect of group, F(1, 24)=0.915, n.s., a non-significant main effect of pre-sample session treatment, F(1, 24)=0.047, n.s., and a non-significant group x pre-sample treatment interaction effect F(1, 24)=1.908, n.s. A similar pattern of results was revealed from the analysis of velocity during the sample session, no significant main effect of group, F(1, 24)=0.930, n.s., of treatment, F(1, 24)=0.042, n.s., and no significant interaction of group x pre-sample treatment, F(1, 24)=1.923, n.s. These results suggest that the attention and motivation to explore the objects during the sample session was not affected by acute drug administration or the prior exposure to KET or SAL.

Test Session. In the 15.0 mg/kg group, as indicative of poor memory, KET mice that received pre-sample session VEH spent significantly less time exploring the novel object compared to SAL mice that received pre-sample session VEH. However, KET mice that received pre-sample CyPPA (15 mg/kg) exhibited significantly greater preference for exploring the novel object during the test session compared to KET mice that had received pre-sample session VEH, indicating a rescue of recognition memory (see FIG. 1). A two-factor ANOVA (group: SAL vs. KET; pre-sample treatment: VEH vs. CyPPA) on discrimination ratio measures revealed a significant group x pre-sample treatment interaction effect, F(1, 24)=17.427, p<0.001 (FIG. 1). The analysis revealed a non-significant main effect of group, F(1, 24)=2.919, n.s.; and a non-significant main effect of pre-sample session treatment, F(1,24)=0.675, n.s. Holm-Sidak pairwise multiple comparison tests, following the significant interaction, yielded significant differences in treatment, p<0.05, within SAL: SAL/VEH vs. SAL/CyPPA; KET: KET/VEH vs. KET/CyPPA; VEH: SAL/VEH vs. KET/VEH. Mann Whitney tests revealed that SAL/VEH-(Mdn=0.617), SAL/CyPPA-(Mdn=0.381), and KET/CyPPA-treated mice, (Mdn=0.191), U=0.000, all exhibited discrimination ratio scores that were significantly above chance (Mdn=0.000), p<0.001. However, the discrimination ratio scores of KET/VEH-treated mice (Mdn=0.188) was not greater than chance (Mdn=0.000), U=14.000, n.s., consistent with impaired memory in this mouse model of schizophrenia-like behavior.

A two-factor ANOVA on total object exploration during test session revealed no significant main effect of group, F(1,24)=3.948. n.s., or pre-sample treatment, F(1, 24)=0.846, n.s., but a significant group x pre-sample treatment interaction effect, F(1, 24)=7.934, p<0.010. Holm-Sidak pairwise comparison revealed a significant difference in treatment, p<0.05, within KET: KET/VEH (M=36.303) vs. KET/CyPPA (M=22.274), and in group within CyPPA: SAL/CyPPA (M=34.878) vs. KET/CyPPA (M=22.274).

In the 7.5 mg/kg group, a one-way ANOVA revealed a significant effect between KET vs. SAL groups, F(2, 18)=27.255, p<0.001. Post-hoc Holm-Sidak tests found significance between SAL/VEH vs. KET/VEH, p<0.001; and SAL/VEH vs. KET/CyPPA, p<0.001; but no significant difference between KET/VEH vs. KET/CyPPA, n.s. Further analysis with Mann Whitney tests revealed that the SAL/VEH (Mdn=0.444), U=0.000, p<0.001, treatment group exhibited a discrimination ratio significantly above chance (Mdn=0.000). A Students t-Test revealed no significant difference between treatment group KET/VEH and chance, t(12)=1.89, n.s.; and a similar finding with a Mann Whitney test in KET/CyPPA (Mdn=0.079), and chance, U=6.000, n.s. A one-way ANOVA on total object exploration during test session revealed no significant difference between groups, F(2, 18)=3.251, n.s.

The results indicate that the KET-mice that received vehicle exhibited an impairment of object memory. The memory impairment exhibited by the KET-mice was not associated with an overall deficit in motivation to explore objects during the sample or test session. KET mice given the SK2 activator CyPPA 15 mg/kg exhibited significantly better object recognition memory performance during the test session compared to KET mice given VEH. This result indicates that the activation of SK2 channels enhanced the memory encoding and consolidation of long-term memory in the KET model of psychosis or schizophrenia-like behavior. These results are consistent with the view that activation of SK2 channel subunits can rescue KET-induced cognitive deficits. In contrast, the SK2 activator CyPPA impaired the encoding and or consolidation of object memory in the SAL control mice, a result consistent with prior reports. These results confirm the impairment of object memory in mice after repeated sub-chronic treatment of KET, and a that while there was a significant improvement in object memory within the KET-mice that received high dose CyPPA IP (15.0 mg/kg) this was not demonstrated in KET-treated mice that received low dose CyPPA IP (7.5 mg/kg). The finding that KET/CyPPA mice performed of a significantly worse than difference in test session discrimination ratio between the SAL/VEH mice and KET/CyPPA mice suggests that a low dose of the SK2 activator does is not sufficient to produce partial rescue of hippocampal memory in the KET model.

### Example 2. Intrahippocampal Infusion of CyPPA Rescues Recognition Memory in the KET Model

Long-term object recognition memory is dependent on the hippocampus, a brain region in the memory circuit that is rich in SK2 channels. To determine whether the CyPPA-mediated rescue of memory in KET mice involved activation of SK2 channel subunits in the hippocampus, a second cohort of KET mice received local bilateral microinfusion of CyPPA (1 μg/μl) or Cremaphor (i.e., VEH) into the CA1 region of dorsal hippocampus prior to the sample session. The object recognition task was conducted identical to that described above. The results confirmed the IP CyPPA study, activation of hippocampal SK2 channels in KET mice by pre-sample intra-CA1 infusion of CyPPA, led to the rescue of object memory 24-h later. KET mice that received pre-sample intra-CA1 infusion of VEH exhibited impaired object memory 24-h later, compared to SAL mice, consistent with the cognitive deficits observed in KET mice in our previous studies.

Local Infusion of SK2 Activator in the CA1 region of Hippocampus. Mice underwent stereotaxic surgery to implant indwelling 26 g guide cannulae above the CA1 region of hippocampus, and then were allowed to recover for 7-10 days. For intrahippocampal microinfusion SAL mice received bilateral (0.35 μl/side, 0.334 μl/min) intra-hippocampal treatment with Cremaphor VEH 5 min prior to the sample session. KET mice received bilateral intra-hippocampal with either CyPPA (1 μg/μl in 0.9% saline), or the Cremaphor VEH, 5 m prior to the sample session.

### Results

Histological analyses revealed correct placement of cannulae within the CA1 in 41 of 47 mice. The data of 6 mice were removed due to inaccurate cannula placement; 4 cannulae were positioned anterior to the desired target, and 2 were unilateral.

Sample Session. A Kruskal-Wallis one-way ANOVA on rank on total object exploration time revealed no significant difference between groups/pre-sample treatment condition, SAL/VEH (Mdn=64.451 s), KET/VEH (Mdn=60.030 s), KET/CyPPA (Mdn=62.190 s), H (2)=1.968, n.s. Similar analyes yielded no significant difference between groups for distance traveled, SAL/VEH (Mdn=2404.640), KET/VEH (Mdn=2452.78), KET/CyPPA (Mdn=2504.32), H (2)=0.060, n.s.; and velocity, SAL/VEH (Mdn=7.997), KET/VEH (Mdn=8.443), KET/CyPPA (Mdn=8.3), H (2)=0.0853, n.s. The results suggest that the local intrahippocampal infusion of the SK2 activator did not affect the motivation to explore the sample objects.

Test Session. Twenty-four hours after the sample session mice received an object memory test exactly as described in the previous section. SAL mice that received intra-CA1 VEH pre-sample session exhibited significantly greater preference for exploring the novel object compared to either KET/VEH- or KET/CyPPA-treated mice. Mice that received KET/CyPPA-demonstrated significantly greater exploration of the novel test object than did the KET/VEH-treated mice, indicating a rescue of object memory following the local CA1 infusion of the SK2 activator, confirming the previously described IP study. A one-way ANOVA on discrimination ratio scores revealed a significant effect of group/treatment condition, F(2, 20)=38.484, p<0.001. Post-hoc Holm-Sidak tests found significance between SAL/VEH vs. KET/VEH, p<0.001; SAL/VEH vs. KET/CyPPA (M=0.354), p<0.001; and KET/VEH vs. KET/CyPPA, p<0.001 (FIG. 2). Further analysis with Mann Whitney tests revealed that treatment groups SAL/VEH (Mdn=0.651), U=0.000, p<0.001; and KET/CyPPA (Mdn=0.398), U=18.000, p<0.038; exhibited discrimination ratios significantly above chance (Mdn=0.000). There was no significant difference between KET/VEH (Mdn=0.046) and chance, U=36.000, n.s. A one-way ANOVA on total object exploration during test session revealed a significant difference between groups, F(2, 33)=3.941, p=0.029. Holm-Sidak pairwise comparison demonstrated a significant difference in SAL/VEH vs. KET/VEH, p=0.030. It appears that KET mice that received either pre-sample VEH or CyPPA were less motivated to explore the objects compared to the SAL mice. However, mice that received KET/CyPPA still displayed significantly greater object memory than KET/VEH mice as demonstrated in the test session, indicating that the reduction of object exploration caused by KET did not affect the ability of the SK2 activator to rescue the memory.

These results confirm the impairment of object memory in mice after chronic treatment of KET, and the significant improvement in object memory within the KET mice that received CyPPA. The intrahippocampal infusion of the SK2 activator before training induced a partial, albeit significant, rescue of object recognition memory in the KET mice. This result suggests that systemically administered CyPPA may be activating SK2 channels in other regions beyond the CA1 as well to produce a more complete rescue of object memory.

### Example 3. Chlorzoxazone Object Recognition Memory

Here, the influence of the SK channel activator, chlorzoxazone (CZX) was tested on object recognition memory. Object recognition sample and test sessions were conducted identical to example 1. SAL mice given CZX before the sample session exhibited impaired object memory during the test session 24 hours later as compared to SAL mice given VEH. That the SK channel activator impaired object memory in healthy control mice is consistent with previous reports (Vick et al., 2010; Mckay et al., 2012). However, KET mice given pre-sample CZX exhibited significantly better performance in the object memory task compared to that of KET mice given VEH. This result indicates that CZX rescued a cognitive deficit characteristic of the KET mouse model of psychosis or schizophrenia-like behavior.

### Methods

SK Administration. Cohorts of SAL and KET mice received a systemic injection of CZX (5.0 mg/kg) or DMSO (4%, VEH) 30 min prior to the sample session.

### Results

Sample Session. Mice were given 10 min to acquire the object exploration criterion. Two-factor ANOVAs revealed that pre-sample session treatment did not affect attention or motivation to explore the sample objects within either group. Distance traveled and velocity was non-significant between groups.

Test Session. A two-factor ANOVA with a log transform on test session total object exploration time demonstrated a non-significant main effect of group, KET vs. SAL, F(1, 28)=2.472, n.s. of pre-sample treatment, VEH vs. CZX, F(1, 28)=0.395, n.s.; and a significant pre-sample treatment x time bin interaction, F(1, 28)=9.641, p=0.004. Holm-Sidak pairwise comparison revealed a significant difference in total object exploration between SAL/VEH vs. SAL/CZX, p=0.020; and in SAL/VEH vs. KET/VEH-mice, p=0.002.

The results of a t-test revealed that all groups that received VEH pre-sample exhibited discrimination ratio scores that were significantly different from chance (Mdn=0.000): SAL×VEH (Mdn=0.463), U=0.000, p<0.001; KET×VEH (Mdn=0.187), U=0.000, p<0.001; SAL×CZX (Mdn=0.316), U=0.000, p<0.001; and KET×chlorzoxazone, t(16)=10.238, p<0.001 (FIG. 3).

The results indicate that the pre-sample administration of systemic CZX to KET mice improved object recognition memory compared to the KET mice given vehicle. This result is consistent with the view that SK channel activating drugs have efficacy in relieving cognitive impairments in a mouse model of schizophrenia. Further, the results suggest that administration of the nonselective SK activator CZX rescued object memory performance in the KET mice comparable to the results found for the SK2 specific activator, CyPPA as described above.

### Example 4. CyPPA Trace Fear Conditioning

The cognitive deficits associated with schizophrenia include impairments in attention and temporal memory; that is, memory for the timing between relevant stimuli. Long-term temporal memory is also dependent upon the hippocampus and both attention and temporal memory can be assessed in rodents with a trace fear conditioning protocol.

### Materials and Methods

Systemic Administration of the SK2 Activator. Two groups of KET mice and two groups of SAL mice were randomly assigned to receive either VEH or CyPPA (15.0 mg/kg) IP 30 min prior to a trace fear conditioning session.

Trace fear conditioning was performed using a MED Associates Near-Infrared Video Fear Conditioning System (Georgia, VT) composed of four identical rectangular conditioning chambers (30.5 cm by 24.1 cm by 21 cm) constructed of brushed aluminum side walls and clear Plexiglas front, back and top walls. Each chamber was illuminated by an overhead white house light and an infrared light. An audio speaker attached to the right-side wall of each chamber delivered the tone stimuli. The chamber floor was constructed of parallel stainless-steel rods (36 rods, 3.2 mm dia, 7.9 mm apart) designed for mice, and connected to a scrambled shock generator. Each conditioning chamber was housed inside a larger sound-attenuating cabinet with a ventilation fan in the right-side wall used to provide background noise. A near-infrared FireWire video camera, mounted on the left front door of each noise-attenuating cabinet, was used to acquire mouse behavior. Before each trial, the chamber floors were cleaned thoroughly with a 70% ethanol solution, then with 1% LiquiNox (White Plains, New York) to reduce olfactory cues.

Freezing responses were recorded and automatically scored during the conditioning session to assess the strength of fear memory. The automatic software program operationally defined freezing as the duration of time, greater than 0.6 s, during which fewer than 20 pixels of each video frame were detected to have changed. The video capture rate was set at 30 fps which resulted in freezing being recorded computationally after less than 20 pixels of motion per frame over the time course of 18 frames. All final freezing responses were hand scored to ensure accuracy.

Trace Fear Conditioning Session. Mice received a systemic injection of CyPPA or VEH and then were placed individually into empty holding cages. Thirty min later, mice were placed individually into a distinct conditioning chamber with an odor cue. One min after placement in the chamber, a 5000 Hz, 90 dB, 15-s tone conditioned stimulus (CS) was presented followed by a 15-s stimulus-free delay or gap before the presentation of a 1-s, 0.5 mA foot shock unconditioned stimulus (US). The CS-US pairing was repeated 7 more times, with a 120-s interval was imposed between CS presentations. (FIG. 4A). Mice were removed from the chamber 60 s. after the presentation of the 8th US.

Trace Fear Memory Tone Test. To assess retention of trace fear memory after a 24-hour delay, mice received a mock IP injection before being placed in the polycarbonate cage. After 30 min, mice were placed into modified conditioning chambers and after a 60 s. delay, were presented with the identical tone CS as in the conditioning session. A second and third CS was presented following a 120-s interstimulus interval (FIG. 4.B). Mice were removed 30 s. after termination of the last CS. The context of chamber was modified before the tone test by installing an inverted V-shaped Plexiglas insert to change the geometry of the side walls and ceiling, a floor shield, 1% acetic acid, and lighting filter.

Statistical Analysis. Percent freezing scores for each mouse during the trace conditioning session were first analyzed by a repeated-measures ANOVA with the between-subjects factors being group (SAL or KET), and treatment(CyPPA or VEH) and the within-subjects factor being time bin (the first 60 s. pre-CS period, and each 15 s. CS period or CS post 1 (the 15 s. following the CS) on % freezing scores. Significant interaction effects and time bin effects were followed with post-hoc multiple comparisons tests. For the tone test session, % freezing scores were analyzed using a repeated-measures ANOVA with the between-subjects factor being group (SAL or KET), and treatment(CyPPA VEH) and the within-subjects factor being time bin (the first 30 s. pre-CS period, the 15 s. CS period and 15s post CS time periods, with post multiple comparisons tests where appropriate. To examine whether KET differentially influenced the perception of the aversive foot shock stimulus, an activity burst ratio was computed for each mouse by dividing the motion score for the first shock interval, 1 s, +1 s. post shock by the motion score for a 2-s interval from the initial 60 s. of the conditioning session (Mean shock/mean no shock). A two-factor (group, treatment) ANOVA, or one-way if not normally distributed, was conducted to analyze treatment differences in the mean activity burst.

Trace fear conditioning involves the imposition of a temporal gap between the termination of the CS and the onset of the aversive US stimulus. An association between the CS and the US develops across this temporal gap. During trace fear conditioning mice gradually acquire an appropriately timed anticipatory conditioned freezing response. The strength and accuracy of this temporally guided fear memory can then be assessed during a test session, often presented 24 hours after trace conditioning. The expectation is that the appropriately conditioned mouse will express the conditioned freezing response during the trace interval, and not during the CS period. Thus, the task requires the mice to exhibit a discriminative conditioned response.

The % of freezing was analyzed across group and treatment between: % freezing during all test session CS's (CS1+CS2+CS3/3); the mean % freezing during the first 15 s. of trace period following all CSs ((post 1 CS1+post 1 CS2+post 1 CS3)/3); the mean % freezing during both the 15 s. trace periods following the CS (post 1 CS+post 2 CS)/2); and the mean % freezing during all the trace periods ((post 1 CS1+post 2 CS1)+ (post 1 CS2+post 2 CS2)+ (post 1 CS3+post 2 CS3))/3). To better capture the acquisition of an appropriately timed freezing response by the mice, a mean discrimination ratio of freezing during the trace interval minus freezing was computed for each mouse by finding the difference score of mean % freezing during the trace intervals minus mean % freezing during the CS intervals, and then dividing the result by the mean % freezing during the trace intervals and the CS intervals. A discrimination ratio score greater than 0 would indicate a preference for expressing more freezing during the trace interval than during the CS interval. A score of 0 would indicate a lack of discrimination.

Additional analyses were conducted to evaluate the treatment effect on the acquisition of anticipatory freezing responses during the trace conditioning session to determine whether the SK2 activator influenced attention or learning.

### Results

Trace Fear Conditioning Tone Test. The SAL mice given VEH exhibited significantly greater % freezing during the 15-s trace intervals compared to that during the 15-s CS intervals, than both SAL mice given CyPPA and KET mice given VEH, suggesting impaired attention and or learning in the latter groups. KET mice given CyPPA demonstrated significantly greater % freezing during the 15-s trace intervals than during the 15-s CS intervals compared to KET mice given VEH, indicating a rescue of the attention or learning deficit. As shown in FIG. 4, a two-factor ANOVA on discrimination ratio of % freezing (mean of all 15-s trace intervals minus the mean of all 15-s CS intervals)/(mean of all 15-s trace intervals+mean of all 15-s CS intervals) yielded no significant main effect of group, F(1, 34)=1.21, n.s.; or preconditioning treatment, F(1, 34)=0.43, n.s.; but a significant group x preconditioning treatment interaction effect, F(1, 34)=7.98, p=0.008. Holm-Sidak pairwise comparisons tests revealed significant differences in % freezing between preconditioning treatment within KET: KET/VEH vs. KET/CyPPA, p=0.02, and a significant difference in group within VEH: SAL/VEH vs. KET/VEH, p=0.01. Mann Whitney tests revealed a significant difference between chance (Mdn=0.00): vs. SAL/VEH (Mdn=0.28), U=9.000, p=0.003; vs. SAL/CyPPA (Mdn=0.13), U=10.000, p=0.001; and vs. KET/CyPPA (Mdn=0.21), U=10.000, p=0.001. There was no significant difference between KET/VEH (Mdn=0.01) and chance (Mdn=0.00), U=27.000, n.s.

These results reveal that the systemic administration of CyPPA improved the acquisition of the conditioned freezing response in KET-mice in a hippocampal-dependent trace conditioning protocol. The same dose of the SK2 activator impaired acquisition of the conditioned freezing response in the SAL-mice. Attention is required for successful association of stimuli in trace fear conditioning (Brzózka & Rossner, 2013; Han et al., 2003). The improved test session performance of KET mice that received CyPPA may be a result of improved attention seen during conditioning and facilitated encoding and consolidation of temporal memory.

### Example 5. Chloroxazone Trace Fear Memory

Here, the influence of the SK channel activator, CZX was tested on trace fear memory. Trace fear conditioning, conditioning and test sessions were conducted identical to example 4. SAL mice given CZX before the conditioning session exhibited impaired trace fear conditioning memory during the test session 24 hours later as compared to SAL mice given VEH. However, KET mice given pre-conditioning CZX exhibited significantly better performance in the trace fear conditioning memory task compared to that of KET mice given VEH. This result indicates that CZX rescued a cognitive deficit characteristic of the KET mouse model of psychosis or schizophrenia-like behavior.

### Methods

SK Administration. Cohorts of SAL and KET mice received a systemic injection of CZX (5.0 mg/kg) or DMSO (4%, VEH) 30 min prior to the sample session. The cognitive deficits associated with schizophrenia include impairments in attention and temporal memory; that is, memory for the timing between relevant stimuli. Long-term temporal memory is also dependent upon the hippocampus and both attention and temporal memory can be assessed in rodents with a trace fear conditioning protocol.

### Materials and Methods

Systemic Administration of the SK2 Activator. Two groups of KET mice and two groups of SAL mice were randomly assigned to receive either VEH or CZX (5.0 mg/kg) IP 30 min prior to a trace fear conditioning session.

### Results

Trace Fear Conditioning Tone Test. The SAL mice given VEH exhibited significantly greater % freezing during the 15-s trace intervals compared to that during the 15-s CS intervals, than both SAL mice given CZX and KET mice given VEH, suggesting impaired attention and or learning in the latter groups. KET mice given CZX demonstrated significantly greater % freezing during the 15-s trace intervals than during the 15-s CS intervals compared to KET mice given VEH, indicating a rescue of the attention or learning deficit. As shown in Figure XX, a two-factor ANOVA on discrimination ratio of % freezing (mean of all 15-s trace intervals minus the mean of all 15-s CS intervals)/(mean of all 15-s trace intervals+mean of all 15-s CS intervals) yielded no significant main effect of group, F(1, 20)=2.279, n.s.; but a significant effect in preconditioning treatment, F(1, 20)=9.303, p=0.006; and a significant group x preconditioning treatment interaction effect, F(1, 20)=4.828, p=0.040. Holm-Sidak pairwise comparisons tests revealed significant differences in % freezing between preconditioning treatment within KET: KET/VEH vs. KET/CZX, p<0.05, a significant difference in group within VEH: SAL/VEH vs. KET/VEH, p<0.05, and a significant difference in group within CZX: SAL/CZX vs. KET/CZX, p<0.05. There was no significant difference between preconditioning treatment within SAL: SAL/VEH vs SAL/CZX, n.s.

These results reveal that the systemic administration of CZX improved the acquisition of the conditioned freezing response in KET-mice in a hippocampal-dependent trace conditioning protocol.

### CONCLUSIONS

The effects of chlorzoxazone (CZX), a non-selective SK activator and CyPPA, an SK2 subunit activator were examined in distinct cohorts of SAL and KET mice. The experiments yielded several important results. First, KET mice given CZX or CyPPA spent significantly more time exploring the novel object during the object recognition test session. These results indicate that CZX and CyPPA rescued object recognition memory in the KET mice. Second, in the trace conditioning protocol to test memory for timing, the KET mice administered CyPPA froze significantly more during trace periods of the trace conditioning session, than during CS periods. However, during the tone test, KET mice given CyPPA mice performed equivalently to KET mice given VEH. This pattern of results suggests that the SK2 activator selectively improved acquisition of temporal fear memory in KET-mice but did not affect its subsequent retrieval. The prolonged deficit of object memory observed in the KET mice suggests significant compromise of hippocampal memory circuits. KET mice that received CyPPA or CZX prior to the sample session, exhibited a significant preference for exploring the novel test object over the familiar one, compared to KET mice given VEH. These results indicate that enhancement of SK channel function can significantly alleviate the impairment of memory in KET mice. Interestingly, the selective infusion of CyPPA into the CA1 region of the hippocampus recapitulated the systemic rescue effect on object memory in the KET mice. These results suggest that the CyPPA-induced improvement in encoding and/or consolidation of object memory in KET mice is due in part to the activation of SK2 channels within the CA1 region of the hippocampus. The results also confirmed that pre-sample session administration of systemic CyPPA impaired object memory in SAL mice, compared to controls. These results confirm and extend previous results indicating that increasing SK channel activity impairs hippocampal dependent memory in healthy control mice (Vick et al., 2010; Mckay et al., 2012; Hammond et al., 2006; Stackman et al., 2008; Criado-Marrero et al., 2014; Blank et al., 2003). Taken together, the results indicate that the same dose of either CyPPA or CZX can impair memory processes in normal healthy mice but improve memory in a KET pharmacological model of schizophrenia-like behavior.

While CZX and CyPPA have not been previously tested in a trace fear protocol, our finding that SAL mice given preconditioning with CZX or CyPPA exhibited impaired freezing compared to SAL mice given VEH, a difference that approached significance, is consistent with previous studies demonstrating that modulation of SK channels with SK activators impairs hippocampal dependent memory in trace fear conditioning and fear extinction protocols. These results indicate that CZX and CyPPA may impair memory in SAL mice but improves hippocampal-dependent memory in a KET pharmacological model of schizophrenia-like behavior. The finding that enhancement of SK channel function improves memory in KET mice is consistent with a report that CyPPA and 1-EBIO alleviated fear memory impairments in a rat model of repeated stress (Atchley et al., 2012).

While the invention has been described in detail and with reference to specific examples thereof, it will be apparent to one skilled in the art that various changes and modifications can be made therein without departing from the spirit and scope thereof.

### REFERENCES

1. Adelman, J P, Maylie, J, & Sah, P. (2012). Small-conductance Ca²⁺-activated K+ channels: form and function. Annu Rev Physiol, 74, 245-269. https://doi.org/10.1146/annurev-physiol-020911-153336
2. Aleman, A, Hijman, R, de Haan, HFE., & Kahn, R S (1999). Memory Impairment in Schizophrenia: A Meta-Analysis. American Journal of Psychiatry, 156 (9), 1358-1366.
3. Alviña, K, & Khodakhah, K (2010). The Therapeutic Mode of Action of 4-Aminopyridine in Cerebellar Ataxia. Journal of Neuroscience, 30 (21), 7258-7268.
4. Arslan, FC, Tiryaki, A, Yildirim, M, Özkorumak, E, Alver, A, Altun, İ, İnce, İ, & Gedikli, Ö (2016). The effects of edaravone in ketamine-induced model of mania in rats. Acta Neurobiol Exp (Wars), 76 (3), 192-198. https://doi.org/10.21307/ane-2017-019
5. Atchley, D., Hankosky, E. R., Gasparotto, K., & Rosenkranz, J. A. (2012). Pharmacological enhancement of calcium-activated potassium channel function reduces the effects of repeated stress on fear memory. Behav Brain Res, 232 (1), 37-43.
6. Balu, DT, Lia, Y, Puhl, M D, Benneyworth, M A, Basu, AC, Takagi, S, Bolshakov, V Y, & Coyle, JT (2013). Multiple risk pathways for schizophrenia converge in serine racemase knockout mice, a mouse model of NMDA receptor hypofunction. PNAS, 110 (26), E2400-E2409.
7. Bangasser, D A, Waxler, D E, Santollo, J, & Shors, T J (2006). Trace conditioning and the hippocampus: the importance of contiguity. J Neurosci, 26 (34), 8702-8706. https://doi.org/10.1523/jneurosci.1742-06.2006
8 Blank, T, Nijholt, I, Kye, MJ, Radulovic, J, & Spiess, J. (2003) Small-conductance, Ca²⁺-activated K+ channel SK3 generates age-related memory and LTP deficits. Nature Neurosci, 6 (9), 911-912. https://doi.org/10.1038/nn1101
9 Bolbecker, A R, Mehta, C, Johannesen, J K, Edwards, CR, O'Donnell, B F, Shekhar, A, Nurnberger, J I, Steinmetz, JE, & Hetrick, WP (2009). Eyeblink conditioning anomalies in bipolar disorder suggest cerebellar dysfunction. Bipolar Disorder International Journal of Psychiatry and Neurosciences, 11 (1), 19-32.
10. Bond, C T, Herson, P S, Strassmaier, T, Hammond, R, Stackman Jr, R. W, Maylie, J, & Adelman, JP (2004). Small Conductance Ca<sup>2+</sup>-Activated K<sup>+</sup> Channel Knock-Out Mice Reveal the Identity of Calcium-Dependent Afterhyperpolarization Currents. The Journal of Neuroscience, 24 (23), 5301. https://doi.org/10.1523/JNEUROSCI.0182-04.2004
11. Bowden, SEH, Fletcher, S, Loane, D J, & Marrion, N V (2001). Somatic colocalization of rat SK1 and D class (Ca (v) 1.2) L-type calcium channels in rat CA1 hippocampal pyramidal neurons. Journal of Neuroscience, 21, 1-6.
12. Brini, M, & Carafoli, E (2009). Calcium pumps in health and disease. Physiol. Rev., 89, 1341-1378.
13. Broadbent, N J, Gaskin, S, Squire, L R, & Clark, RE (2010). Object recognition memory and the rodent hippocampus. Learn Mem, 17 (1), 5-11. https://doi.org/10.1101/lm.1650110
14. Brzózka, MM, & Rossner, MJ (2013). Deficits in trace fear memory in a mouse model of the schizophrenia risk gene TCF4. Behav Brain Res, 237, 348-356. https://doi.org/10.1016/j.bbr.2012.10.001
15. Carlsson, A., & Carlsson, M. L. (2006). A dopaminergic deficit hypothesis of schizophrenia: the path to discovery. Dialogues Clin Neurosci., 8 (1), 137-142.
16. Clark, B. A., Monsivais, P., Branco, T., London, M., & Häusser, M. (2005). The site of action potential initiation in cerebellar Purkinje neurons. Nat Neurosci, 8 (2), 137-139. https://doi.org/10.1038/nn1390
17. Clark, R. E., & Squire, L. R. (1998). Classical conditioning and brain systems: The role of awareness. Science, 280 (5360), 77-81.
18. Cohen, S. J., Munchow, A. H., Rios, L. M., Zhang, G., Ásgeirsdóttir, H. N., & Stackman Jr., R. W. (2013). The rodent hippocampus is essential for non-spatial object memory. Current Biology, 23 (17), 1685-1690. https://doi.org/10.1016/j.cub.2013.07.002
19. Cohen, S. J., & Stackman Jr., R. W. (2015). Assessing rodent hippocampal involvement in the novel object recognition task. A review. Behav Brain Res, 285, 105-117. https://doi.org/10.1016/j.bbr.2014.08.002
20. Craddock, N., O'Donovan, M. C., & Owen, M. J. (2005). The genetics of schizophrenia and bipolar disorder: dissecting psychosis. Journal of Medical Genetics, 42 (3), 193-204.
21. Criado-Marrero, M., Santini, E., & Porter, J. T. (2014). Modulating fear extinction memory by manipulating SK potassium channels in the infralimbic cortex. Frontiers in behavioral neuroscience, 8, 96-96. https://doi.org/10.3389/fnbeh.2014.00096
22. Dimitriadi, M., Kye, M. J., Kalloo, G., Yersak, J. M., Sahin, M., & Hart, A. C. (2013). The Neuroprotective Drug Riluzole Acts via Small Conductance Ca²⁺-Activated K+ Channels to Ameliorate Defects in Spinal Muscular Atrophy Models. Journal of Neuroscience, 33 (15), 6557-6562.
23. Durr, A. (2010). Autosomal dominant cerebellar ataxias: polyglutamine expansions and beyond. Lancet Neurol., 9 (9), 885-894.
24. Faber, E. S., Delaney, A. J., & Sah, P. (2005). SK channels regulate excitatory synaptic transmission and plasticity in the lateral amygdala. Nature Neuroscience, 8, 635-641.
25. Gazal, M., Kaufmann, F. N., Acosta, B. A., Oliveira, P. S., Valente, M. R., Ortmann, C. F., Sturbelle, R., Lencina, C. L., Stefanello, F. M., Kaster, M. P., Reginatto, F. H., & Ghisleni, G. (2015). Preventive Effect of Cecropia pachystachya Against Ketamine-Induced Manic Behavior and Oxidative Stress in Rats. Neurochem Res, 40 (7), 1421-1430. https://doi.org/10.1007/s11064-015-1610-5.
26. Ghedim, F. V., de B Fraga, D., Deroza, P. F., Oliveira, M. B., Valvassori, S. S., Steckert, A. V., Budni, J., Dal-Pizzol, F., Quevedo, J., & Zugno, A. I. (2012). Evaluation of Behavioral and Neurochemical Changes Induced by Ketamine in Rats: Implications as an Animal Model of Mania. J Psychiatr Res, 46 (12), 569-575.
27. Goff, D. C., Hill, M., & Barch, D. (2011). The Treatment of Cognitive Impairment in Schizophrenia. Pharmacol Biochem Behav, 99 (2), 245-253.
28. Hammond, R. S., Bond, C. T., Strassmaier, T., Ngo-Anh, T. J., Adelman, J. P., Maylie, J., & Stackman Jr., R. W. (2006). Small-conductance Ca²⁺-activated K+Channel Type 2 (SK2) Modulates Hippocampal Learning, Memory, and Synaptic Plasticity. Journal of Neuroscience, 26 (6), 1844-1853. https://doi.org/10.1523/jneurosci.4106-05.2006
29. Hammond, R. S., Tull, L. E., & Stackman Jr., R. W. (2004). On the delay-dependent involvement of the hippocampus in object recognition memory. Neurobiology of Learning and Memory, 82 (1), 26-34.
30. Han, C. J., O'Tuathaigh, C. M., van Trigt, L., Quinn, J. J., Fanselow, M. S., Mongeau, R., Koch, C., & Anderson, D. J. (2003). Trace but not delay fear conditioning requires attention and the anterior cingulate cortex. Proc Natl Acad Sci USA, 100 (22), 13087-13092. https://doi.org/10.1073/pnas.2132313100
31 Hashimoto, K. (2006). The NMDA Receptor Hypofunction Hypothesis for Schizophrenia and Glycine Modulatory Sites on the NMDA Receptors as Potential Therapeutic Drugs. Clinical Psychopharmacology and Neuroscience, 4 (1), 3-10.
32 Hauser, M. J., Isbrandt, D., & Roeper, J. (2017). Disturbances of novel object exploration and recognition in a chronic ketamine mouse model of schizophrenia. Behav Brain Res, 332, 316-326. https://doi.org/10.1016/j.bbr.2017.06.013
33. Hou, Y., Zhang, H., Xie, G., Cao, X., Zhao, Y., Liu, Y., Mao, Z., Yang, J., & Wu, C. (2013). Neuronal injury, but not microglia activation, is associated with ketamine-induced experimental schizophrenic model in mice. Prog Neuropsychopharmacol Biol Psychiatry, 45, 107-116. https://doi.org/10.1016/j.pnpbp.2013.04.006
34. Kolomeets, N. S., Orlovskaya, D. D., Rachmanova, V. I., & Uranova, N. A. (2005). Ultrastructural alterations in hippocampal mossy fiber synapses in schizophrenia: a postmortem morphometric study. Synapse, 57 (1), 47-55. https://doi.org/10.1002/syn.20153
35. Kolomeets, N. S., Orlovskaya, D. D., & Uranova, N. A. (2007). Decreased numerical density of CA3 hippocampal mossy fiber synapses in schizophrenia. Synapse, 61 (8), 615-621. https://doi.org/10.1002/syn.20405
36. Kshatri, A. S., Gonzalez-Hernandez, A., & Giraldez, T. (2018). Physiological Roles and Therapeutic Potential of Ca²⁺ Activated Potassium Channels in the Nervous System. Frontiers in Molecular Neuroscience, 11, 258.
37. Lam, J., Coleman, N., Lourdes, A., Garing, A., & Wulff, H. (2013). The Therapeutic Potential of Small-Conductance KCa2 Channels. Expert Opin Ther Targets, 17 (10), 1203-1220.
38. Lidow, M. S. (2003). Calcium signaling dysfunction in schizophrenia: a unifying approach. Brain Research Review, 43, 70-84.
39. Lin, M. T., Lujan, R., Watanabe, M., Adelman, P., & Maylie, J. (2008). SK2 channel plasticity contributes to LTP at Schaffer collateral-CA1 synapses. Nat Neurosci, 11, 170-177.
40. Lin, M. T., Lujan, R., Watanabe, M., Frerking, M., Maylie, J., & Adelman, J. P. (2010). Coupled activity-dependent trafficking of synaptic SK2 channels and AMPA receptors. The Journal of Neuroscience, 30 (35), 11726-11734.
41. Lisek, M., Ferenc, B., Studzian, M., Pulaski, L., Guo, F., Zylinska, L., & Boczek, T. (2017). Glutamate Deregulation in Ketamine-Induced Psychosis-A Potential Role of PSD95, NMDA Receptor and PMCA Interaction. Front. Cell. Neuroscience.
42. Losin, S., & Mckean, C. M. (1966). Chlorzoxazone (paraflex) in the treatment of severe spasticity. Developmental Medicine and Child Neurology, 8 (6), 768-769.
43. Lyon, L., Saksida, L. M., & Bussey, T. J. (2012). Spontaneous object recognition and its relevance to schizophrenia: a review of findings from pharmacological, genetic, lesion and developmental rodent models. Psychopharmacology (Berl), 220 (4), 647-672. https://doi.org/10.1007/s00213-011-2536-5
44. Marenco, S., Weinberger, D. R., & Schreurs, B. G. (2003). Single-cue delay and trace classical conditioning in schizophrenia. Biological Psychiatry, 53 (5), 390-402.
45. McEchron, M. D., Bouwmeester, H., Tseng, W., Weiss, C., & Disterhoft, J. F. (1998). Hippocampectomy disrupts auditory trace fear conditioning and contextual fear conditioning in the rat. Hippocampus, 8, 638-646.
46. McEchron, M. D., Tseng, W., & Disterhoft, J. F. (2003). Single Neurons in CA1 Hippocampus Encode Trace Interval Duration during Trace Heart Rate (Fear) Conditioning in Rabbit. The Journal of Neuroscience, 23 (4), 1535. https://doi.org/10.1523/JNEUROSCI.23-04-01535.2003
47. Mckay, B. M., Oh, M. M., Galvez, R., Burgdorf, J., Kroes, R. A., Weiss, C., Adelman, J. P., Moskal, J. R., & Disterhoft, J. F. (2012). Increasing SK2 channel activity impairs associative learning. Journal of Neurophysiology, 108 (3), 863-870. https://doi.org/10.1152/jn.00025.2012
48. Miller, R. G., Mitchell, J. D., & Moore, D. H. (2012). Riluzole for amyotrophic lateral sclerosis (ALS)/motor neuron disease (MND). Cochrane Database Syst Rev., 14 (3).
49. Mourre, C., Fournier, C., & Soumireu-Mourat, B. (1997). Apamin, a blocker of the calcium-activated potassium channel, induces neurodegeneration of Purkinje cells exclusively. Brain Research, 778, 405-408.
50. Nair, A B. and Jacob, S. (2016). A simple practice guide for dose conversion between animals and human. J Basic Clin Pharma, 7:27-31.
51. NAMI. (2020). https://doi.org/https://www.nami.org/About-Mental-Illness/Mental-Health-Conditions/Schizophrenia.
52. Ngo-Anh, T. J., Bloodgood, B. L., Lin, M., Sabatini, B. L., Maylie, J., & Adelman, J. P. (2005). SK channels and NMDA receptors form a Ca²⁺-mediated feedback loop in dendritic spines. Nature Neuroscience, 8, 642-649.
53. Nikiforuk, A., & Popik, P. (2014). Ketamine prevents stress-induced cognitive inflexibility in rats. Psychoneuroendocrinology, 40, 119-122.
54. Quinn, J. J., Oommen, S. S., Morrison, G. E., & Fanselow, M. S. (2002). Post-training excitotoxic lesions of the dorsal hippocampus attenuate forward trace, backward trace, and delay fear conditioning in a temporally specific manner. Hippocampus, 12 (4), 495-504. https://doi.org/10.1002/hipo.10029
55. Ragland, J. D., Layher, E., Hannula, D. E., Niendam, T. A., Lesh, T. A., Solomon, M., & Ranganath, C. (2017). Impact of schizophrenia on anterior and posterior hippocampus during memory for complex scenes. NeuroImage: Clinical, 13, 82-88.
56. Rajagopal, L., Burgdorf, J. S., Moskal, J. R., & Meltzer, H. Y. (2016). GLYX-13 (rapastinel) ameliorates subchronic phencyclidine- and ketamine-induced declarative memory deficits in mice. Behav Res, 299, 105-110. https://doi.org/10.1016/j.bbr.2015.10.060
57. Rajagopal, L., Huang, M., Michael, E., Kwon, S., & Meltzer, H. Y. (2018). TPA-023 attenuates subchronic phencyclidine-induced declarative and reversal learning deficits via GABAA receptor agonist mechanism: possible therapeutic target for cognitive deficit in schizophrenia. Neuropharmacology, 43, 2468-2477.
58. Reagan-Shaw, S., Nihal, M., & Ahmad, N. (2008). Dose translation from animal to human studies revisited. FASEB journal: official publication of the Federation of American Societies for Experimental Biology, 22 (3), 659-661. https://doi.org/10.1096/fj.07-9574LSF
59. Robinson, T. E., & Becker, J. B. (1986). Enduring changes in brain and behavior produced by chronic amphetamine administration: a review and evaluation of animal models of amphetamine psychosis. Brain Res, 396 (2), 157-198. https://doi.org/10.1016/s0006-8993 (86) 80193-7
60. Schumacher, A., Sivanandan, B., Tolledo, E. C., Woldegabriel, J., & Ito, R. (2016). Different dosing regimens of repeated ketamine administration have opposite effects on novelty processing in rats. Prog Neuropsychopharmacol Biol Psychiatry, 69, 1-10. https://doi.org/10.1016/j.pnpbp.2016.03.007
61. Stackman Jr., R. W., Bond, C. T., & Adelman, J. P. (2008). Contextual memory deficits observed in mice overexpressing small conductance Ca²⁺-activated K+ type 2 (KCa2.2, SK2) channels are caused by an encoding deficit. Learn Mem, 15 (4), 208-213. https://doi.org/10.1101/lm.906808
62. Stackman Jr., R. W., Hammond, R. S., Linardatos, E., Gerlach, A., Maylie, J., Adelman, J. P., & Tzounopoulos, T. (2002). Small conductance Ca²⁺-activated K+ channels modulate synaptic plasticity and memory encoding. Journal of Neuroscience, 1 (22), 10163-10171. https://doi.org/10.1523/jneurosci.22-23-10163.2002
63. Stocker, M. (2004). Ca (2+)-activated K+ Channels: Molecular Determinants and Function of the SK Family. Nat Rev Neurosci, 10, 758-770.
64. Stocker, M., & Pedarzani, P. (2000). Differential distribution of three Ca²⁺-activated K+Ca²⁺-activated K+ channel subunits, SK1, SK2, and SK3, in the adult rat central nervous system. Molecular and Cellular Neuroscience, 15 (5), 476-493.
65. Tsankova, N. M., Berton, O., Renthal, W., Kumar, A., Neve, R. L., & Nestler, E. J. (2006). Sustained hippocampal chromatin regulation in a mouse model of depression and antidepressant action. Nat Neurosci, 9 (4), 519-525. https://doi.org/10.1038/nn1659
66. Tuscher, J. J., Fortress, A. M., Kim, J., & Frick, K. M. (2015). Regulation of object recognition and object placement by ovarian sex steroid hormones. Behav Brain Res, 285, 140-157. https://doi.org/10.1016/j.bbr.2014.08.001
67. Tzounopoulos, T., & Stackman Jr., R. W. (2003). Enhancing Synaptic Plasticity and Memory: A Role for Small-Conductance Ca (2+)-activated K+ Channels. Neuroscientist, 9 (6), 434-439.
68. van Erp, T. G. M., Lesha, T. A., Knowlton, B. J., Bearden, C. E., Hard, M., Karlsgodt, K. H., Shirinyana, D., Rao, V., Green, M. F., Subotnik, K. L., Nuechterlein, K., & Cannon, T. D. (2008). Remember and know judgments during recognition in chronic schizophrenia. Schizophrenia Research, 100 (1-3), 181-190.
69. Vick, K. A., Guidi, M., & Stackman Jr., R. W. (2010). In vivo pharmacological manipulation of small conductance Ca (2+)-activated K (+) channels influences motor behavior, object memory and fear conditioning. Neuropharmacology, 58 (3), 650-659. https://doi.org/10.1016/j.neuropharm.2009.11.008

What is claimed is:

1. A method of treating a cognitive impairment in a subject comprising administering an SK ion channel activator to the subject.

2. The method of claim 1, further comprising alleviating symptoms of the cognitive impairment, wherein the symptoms originate from a condition selected from the group consisting of a) schizophrenia, b) an attention-deficit disorder, c) a memory deficiency, d) a verbal learning deficiency, and e) an executive function deficiency.

3. The method of claim 1, wherein the subject is a mammal.

4. The method of claim 3, wherein the subject is a human.

5. The method of claim 1, wherein the administering step is carried out through an administration technique including at least one of a) intravenous administration, b) oral administration, and c) transdermal administration.

6. The method of claim 5, wherein oral administration is performed through an excipient selected from the group consisting of a pill, dissolvable film, tablet, capsule, and liquid.

7. The method of claim 1, wherein the SK ion channel activator is an SK2 specific activator.

8. The method of claim 1, wherein the SK ion channel activator is a member of the 1,3-benzoxazole drug class.

9. The method of claim 1, wherein the SK ion channel activator is CyPPA.

10. The method of claim 1, wherein the SK ion channel activator is chlorzoxazone.

11. The method of claim 1, wherein the SK ion channel activator is administered in an amount of 1 to 5 times per day.

12. The method of claim 1, wherein the SK ion channel activator is administered in intervals of every 2 to 5 hours.

13. The method of claim 9, wherein CyPPA is administered at a dose of 70.0 mg/kg.

14. The method of claim 10, wherein the chlorzoxazone is administered at a dose in an amount selected from a range of 0.24 mg/kg to 750 mg/kg.

15. The method of claim 10, wherein the chlorzoxazone is administered 3 to 4 times per day.

16. The method of claim 10, wherein the chlorzoxazone is administered 2 to 5 times per day.
